(19) United States
(12) Patent Application Publication
(10) Pub. No.: US 2022/0338580 A1
(43) Pub. Date: Oct. 27, 2022

(54) Wearable device

(71) Applicant: Suunto Oy, Vantaa (FI)

(72) Inventors: Britta Stockinger, Vantaa (FI); Jere Lallo, Vantaa (FI); Atte Kuparinen, Vantaa (FI); Jouni Junes, Vantaa (FI)

(21) Appl. No.: 17/705,427

(22) Filed: Mar. 28, 2022

(30) Foreign Application Priority Data
Apr. 27, 2021 (FI) 20215491

(51) Int. Cl. A41F 9/00; A41D 1/00

(52) U.S. Cl. CPC A41F 9/00; A41D 1/002; A41D 2500/10

(57) ABSTRACT

According to a first aspect of the present disclosure there is provided a wearable strap which includes a strap body knitted from at least a first main yarn and a heat activated reinforcing yarn.

### FIELD

The invention relates to a wearable device with a strap, such as a watch or wrist computer strap or a heart rate sensor chest strap.

### BACKGROUND

Watches, wrist computers, heart rate sensors and other wearable technology are worn, for example, around a user's wrist or chest, using a strap or band. Such straps are typically wrapped around the circumference of a user's wrist, chest or other body member and fastened via a buckle.

Wearable straps made of fabric can be manufactured by knitting. Knitting is a method by which yarn is manipulated to create a textile or fabric. It is used in many types of garments. Knitting may be done by hand or by machine. Knitting creates stitches: loops of yarn in a row, either flat or in the round (tubular). There are usually many active stitches on the knitting needle at one time. Knitted fabric consists of a number of consecutive rows of connected loops that intermesh with the next and previous rows. As each row is formed, each newly created loop is pulled through one or more loops from the prior row and placed on the gaining needle so that the loops from the prior row can be pulled off the other needle without unraveling.

Knitted straps are typically unsuitable for use with a buckle because the structure is often too flimsy. It would therefore be advantageous to provide for a knitted strap construction which is stiff enough for use with a buckle.

### SUMMARY

The present invention is characterized by the appended independent claims.

According to a first aspect of the present disclosure there is provided a wearable strap which includes a strap body knitted from at least a first main yarn and a heat activated reinforcing yarn.

According to a second aspect of the present disclosure there is provided a method for producing such a wearable strap. In the method, a first main yarn is provided, a heat activated reinforcing yarn is provided, the first main yarn and the reinforcing yarn are knitted into a strap body, and the rigidity of the strap body is increased by heating the reinforcing yarn to or beyond the point of heat activation.

One or more embodiments may include one or more features from the following itemized list:
the strap body is three-dimensionally machine-knitted;
the reinforcing yarn is a fusible yarn;
the reinforcing yarn is a thermoplastic yarn;
the reinforcing yarn has a melting point below that of the other yarns making up the strap body;
the reinforcing yarn has a melting temperature between 60 and 180° C.;
the reinforcing yarn content in the knit is 50% or less, particularly 25% or less, preferably less than 25%;
the strap body comprises at least one reinforcement zone, which extends throughout the strap body or covers only a portion of the strap body and in which reinforcement zone the properties of the reinforcement yarn have been altered through application of heat;
the reinforcement zone comprises compression of the reinforcing yarn, through heat activated reduction in length, imposed on the main yarn interlooped with the reinforcing yarn;
the reinforcement zone comprises fusion of the reinforcing yarn into the first main yarn through heat activated melting of the reinforcing yarn;
the strap comprises a major dimension, wherein the strap body has a stiffness about a bending axis, which is co-planar and transverse in respect to the major dimension of extension, to withstand the weight of the strap body about said bending axis without bending;
the strap comprises a major dimension, a minor dimension, which is transverse to the major dimension, and a thickness, which is transverse to both the major dimension and the minor dimension;
the strap has a flexibility along the major dimension that is greater flexibility of the strap along the minor dimension;
the strap body comprises a plurality of eyelets provided along the strap ;
the flexibility of the strap along the major dimension is such to allow for elastic deformation of the strap body by at least the distance between successive eyelets;
the bulk of the strap body is between 700 and 1400 g/m², e.g. between 800 and 1100 g/m², especially between 900 and 1000 g/m²;
the knit forming the strap body of the strap comprises a second main yarn interlooped with the first main yarn and the reinforcing yarn;
the second main yarn is made of a different material than the first main yarn and the reinforcing yarn;
the first main yarn has a rounder cross-sectional shape than the second main yarn;
the second main yarn has a greater durometer value than the first main yarn;
the strap body comprises an inner layer for placement against user's skin during use;
the inner layer is knitted predominantly of the first main yarn;
the inner layer is knitted with a skin contact pattern;
the inner layer is knitted predominantly of the first main yarn into a skin contact pattern;
the strap body comprises an outer layer for placement away from the user's skin during use;
the outer layer is knitted predominantly of a/the second main yarn;
the outer layer is knitted with a façade pattern;
the outer layer is knitted predominantly of a/the second main yarn into façade pattern;
the façade pattern is more porous than the skin contact pattern;
the façade pattern comprises visible pores;
the skin contact pattern is smoother than the façade pattern;
the strap comprises two edge zones at the edges of the strap extending in an longitudinal direction of the strap;
the strap comprises a central zone located between the edge zones;
the elastic modulus of the central zone is greater than that of the edge zones;
the knit forming the strap body of the strap comprises one or more further functional yarn(s), such as silver or reflective yarns;
the strap body comprises a transverse loop hole at a first end of the strap body;
the strap body comprises a transverse buckle hole at a second end of the strap body;
the material or pattern of the yarn defining the transverse hole is stiffer than the material of the yarn forming other parts of the strap body;
the strap comprises a first part comprising a spring bar provided to the transverse loop hole;
the first part comprises a buckle attached to the transverse buckle hole;
the strap comprises a second part comprising a spring bar provided to the transverse loop hole;
the second part comprising a plurality of eyelets provided along the second part;
the second part is configured to cooperate with the buckle;
the knitting is three-dimensional (3D) knitting;
the entire strap body of the strap is produced in one piece by a 3D knitting machine;
the step of increasing the rigidity of the strap body comprises irreversible changing of the physical properties of the reinforcement yarn;
the step of increasing the rigidity of the strap body comprises shrinking the reinforcing yarn so as to compress the interlooped main yarn;
the step of increasing the rigidity of the strap body comprises fusing the reinforcing yarn into the interlooped main yarn,
the step of increasing the rigidity of the strap body comprises both shrinking the reinforcing yarn so as to compress the interlooped main yarn and fusing the reinforcing yarn into the interlooped main yarn;
the method comprises providing a second main yarn and knitting the second main yarn into the knit forming the strap body;
the knitting step comprises knitting two or layers having different patterns or comprising predominantly different main yarns or both having different patterns and comprising predominantly different main yarns,
the method includes installing hardware, such as a buckle and/or a spring bar to the strap body prior to the heat activation step.

Significant benefits may be achieved by means of the present proposal.

By using a heat activated reinforcing yarn in the knit, the strap body may be made stiff enough to enable easy cooperation with a buckle even when using one hand to secure the wearable device to the user's anatomy, e.g. wrist.

Because the knit is made up by a reinforced interlooped yarn network, there is inherent play in the fabric of the body. Upon exposure to tension, the intermediate space between yarns is first consumed thus typically keeping the strap in the elastic domain. Even if the strap would deform due to a sudden pull, the strap may be returned to the original shape by applying a transverse load to the original load that deformed the strap. Accordingly, the knitted body has a good resilience against plastic deformation.

As the flexibility properties of the strap are varied across different sections thereof, the fit may be optimized for optical heart sensor use.

According to one embodiment, the strap is produced by 3D knitting. The wearable strap may thus be directly knitted to the desired shape without cutting out from the knitted band and finishing. As a result, there is no cutting or waste fabric cost, but there is raw material cost in yarn because fabric is being made at the same time as the strap.

By using three-dimensional knitting technique the strap can be customized as desired. For example, the strap can comprise zones with different material properties, such as mechanical or physical properties. Different parts of the parts of the strap may have different thickness, colors, patterns, and/or volume to each other without the need for post-processing after the knitting stage.

Furthermore, additive manufacturing enables fluent shapes, such as smooth attachment points for attachment of the case. Such fluent shapes improve wearing comfort.

Comfort may be also or further improved by knitting the top side of the strap body with a porous pattern for letting air out, which improves breathability, while knitting the bottom side of the strap body, which contacts the skin, relatively flat.

According to an embodiment of the invention the edge zones of the strap are more elastic than central zone located between the edge zones, which improves the adaptation of the strap around the user's body member, such as a wrist or chest. The improved strap adaptation is more convenient to the user. Further, a measuring sensor of the strap or wrist computer/watch can be tightly pressed against the user's skin which enables more reliable and accurate measurement of heart rate or other vital function.

### BRIEF DESCRIPTION OF THE DRAWINGS

In the following certain exemplary embodiments are described in greater detail with reference to the accompanying drawings, in which:

FIG. 1A illustrates a top elevation view of a first part of a wearable strap in accordance with at least some of the embodiments;

FIG. 1B illustrates a top elevation view of a cooperating second part of a wearable strap in accordance with at least some of the embodiments;

FIG. 2A illustrates a bottom elevation view of the first part FIG. 1;

FIG. 2B illustrates a bottom elevation view of the second part of FIG. 1;

FIG. 3A illustrates a side elevation view of the first part of FIG. 1;

FIG. 3B illustrates a side elevation view of the second part of FIG. 1, and

FIG. 4 illustrates a force vs displacement graph of an exemplary 3D knitted strap body.

### EMBODIMENTS

In the present context the term knit refers to a technique and product produced by such technique, where a fabric is made by interlocking loops of yarn with a knitting machine.

In the present context the dimensions of the strap, i.e. the major and minor dimensions, refer to the dimensions, in which the strap extends.

In the following a novel concept of a knitted strap for a terminal device is proposed. The novel concept is based on the idea of a knitted strap body by means of additive manufacturing, three-dimensional (3D) knitting in particular. 3D knitting enables manufacture of multiple layers of fabric in a single manufacturing stage, which affords a degree of freedom in the design and production. Instead of knitting several individual layers of fabric and sewing the layers together, 3D knitting provides for an opportunity to adjust the thickness of the knit by adding new layers of on the existing knit. Further cutting and/or seaming stages may not be required. On the other hand, 3D knitting provides for building in features into the knit that previously required an additional manufacturing stage. Eyelets, for example, no longer need to be produced by means of an extra reductive manufacturing stage. Rather, eyelets may be knitted into the fabric. Accordingly, the number of manufacturing steps and the amount of waste may be minimized.

The body is therefore preferably knitted with a three-dimensional (3D) knitting technique for the purpose of minimizing waste. Known 3D knitting machines typically comprise a control system for controlling the knitting machine. The control system can comprise a computer with suitable software and hardware for controlling the knitting machine. The knitting machine is used to intermesh yarns into loops resulting in a knitted strap. The knitting machine is adapted to integrate different stitch structures and multiple types of yarn directly into the knit structure.

In 3D knitting a design of a fabric structure, such as a strap body in this application, is converted into a CAD knitting program including a grid representing stitches. The CAD specifications of the fabric structure are input to the 3D knitting machine. Thereafter, the knitting machine knits the fabric structure in accordance with the CAD specifications. The CAD system enables design of specific knit structures and allows various yarns to be knitted into complex patterns. According to an embodiment, the entire strap body is produced in one piece by 3D knitting machine.

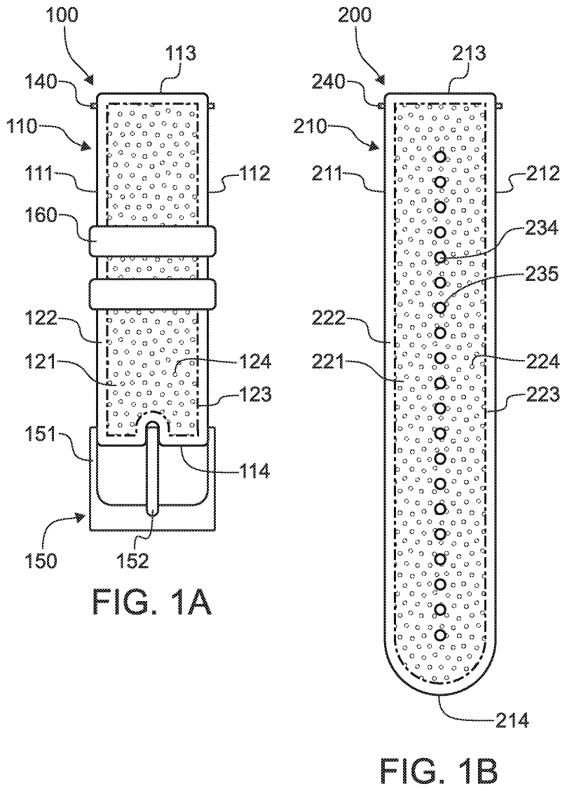

FIGS. 1A to 3B show a two-part strap for a wearable device in a detached configuration. The strap is intended to be attached to a smart watch worn on the user's wrist. The present concept is, however, equally applicable to other terminal devices, such as sensor apparatuses, global positioning apparatuses, etc., as well as to other body parts, such as the head, chest, ankle, etc. The present context is also applicable for producing straps to be worn by animals other than humans. One could, for example, apply the present concept to a GPS harness worn by a hunting dog. Indeed, the herein described strap design could be varied for a host of different terminal devices and hosting body parts.

The illustrated exemplary strap comprises a first part 100 and a second part 200. In the field of wrist watches the first part 100 is referred to as the short part, which includes a buckle 150, and the second part 200 is referred to as the long part, which is intended to interact with the buckle 150 for securing the strap around the user. The strap parts 100, 200 include a knitted body 110, 210 and ancillary items attached thereto, i.e. the textile body and the associated hardware. In the following description features found on both strap parts 100, 200 will have the same reference numeral following the leading number that designates the part, i.e. 1 for the first strap part 100 and 2 for the second strap part 200. For example the reference number suffix 10 for the body is shared across the body parts 100, 200. It follows that description of features of the first strap part 100 is applicable to the second strap part 200, where the second strap part 200 includes the same reference numerals as illustrated in FIGS. 1A to 3B.

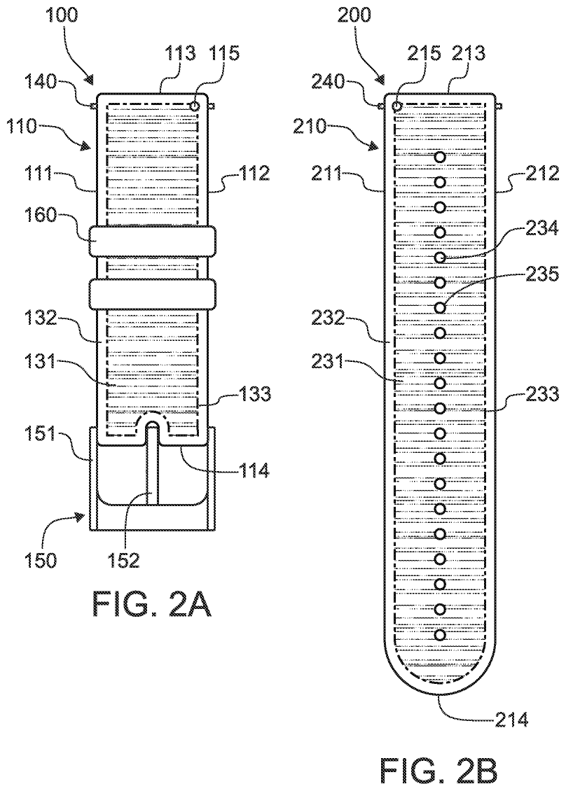

Let us first consider the first strap 100. The first strap part 100 has a knitted body 110. The body 110 is elongated along a major dimension between a first end 113 and a second end 114. The body has a first side 111 and an opposing second side 112 that connect the ends 113, 114 along the major dimension. The distance of the sides 111, 112 defines the width of the body 110 along a minor dimension which is perpendicular to the major dimension. The body 110 also has a thickness in a dimension perpendicular to the plane of FIGS. 1A, 1B, 2A, and 2B.

The same is applicable to the second strap part 200.

Provided to the first end 113 of the first strap part 100 an accessory for attaching the strap part 100 to the wearable device, such as a smart watch. In the illustrated embodiment the accessory is a spring bar 140. The spring bar 140 is installed into a loop hole 116 provided to the first end 113. The loop hole 113 is a transverse hole running through the body 110 in the minor dimension. A release hole 115 is provided on the bottom side of the body 110 shown in FIGS. 2A and 2B. The release tab of the spring bar 140 is accessible through the release hole 115. As will later transpire, the loop hole 113 and release hole 115 are knitted into the body 110 during the additive manufacturing phase of the body 110. As may be appreciated, the transition between the loop hole sections of the body to the rest of the body may be made seamless, which greatly improves user comfort.

The same is applicable to the second strap part 200.

According to the illustrated embodiment the first strap part 100 carries a buckle 150 which is configured to interact with the second strap part 200. The buckle 150 has an open frame 151 for allowing insertion of the second strap part 200 there through. The buckle 150 also has a tang 152 for insertion into the respective eyelets 234 provided to the second strap part 200 in a spaced apart configuration along the major dimension thereof. The frame 151 is attached to the second end 114 of the body 110 with a pin 153 running through a similar or comparable transverse loop hole as provided at the first end 113. The pin 153 may be pin secured to the fabric with an adhesive, screw, or a comparable affixer, a spring bar, or another coupler known in the field.

The first strap part 100 may also feature an accessory for tying excess portions of the second strap part 200 thereto. According to the illustrated embodiment the first strap part 100 comprises loops 160 attached to the bottom side of the body 110 and extending over the top side of the body 110 for receiving the section of the second body 210 extending between the eyelet 234, which accommodates the tang 152, and the second end 114. The first strap 100 could alternatively have fewer or more than two loops 160.

As mentioned above, the additive manufacturing enables the fabrication of several beneficial features into the body. Next the features are described with reference to FIGS. 1A to 3B. It should, however, be noted that the strap may include only one or some of the herein described features.

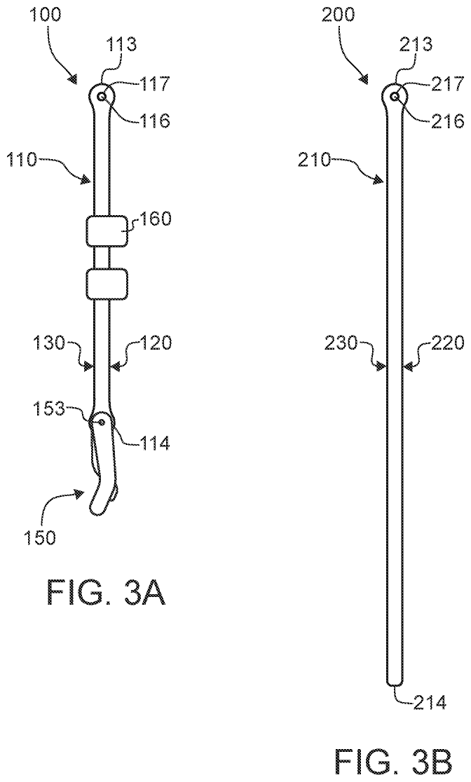

Firstly it may be noted that the top side of the strap body 100 shown in FIG. 1A is different from the bottom side of the strap body 100 shown in FIG. 2A. FIG. 1A shows a relatively porous pattern 121 on the top side compared to the relatively smooth pattern 131 on the bottom side shown in FIG. 2A. The different patterns 121, 132 may be provided by knitting two or more than two layers to the body 110 during the 3D knitting process. As is shown in FIG. 3A, an inner layer 130 is facing the user and an outer layer 120 forms the façade of the strap. The outer layer 120 may include a porous façade pattern 121 with visible pores 124. This means that the inner layer 130 or any intermediate layer (not shown) is visible through the outer layer 120. The pores 124 may be visible with uncompromised vision from a 20 cm distance. The purpose of the relatively porous façade pattern 121 is to provide for a durable and well ventilated façade for the strap.

The skin contact pattern 131 on the inner layer 131 is smoother than the façade pattern 121. Accordingly, the skin contact pattern 131 is a denser knit compared to the façade pattern 121 with fewer and/or shallower protrusion to avoid skin irritation.

The resulting knit structure for the façade pattern 121 and skin contact pattern 131 may be, for example, a single eyelet double faced mesh jacquard.

The top and bottom sides of the strap may exhibit more than one pattern. As is shown in FIGS. 1A and 2A, the façade pattern 121 and the skin contact pattern 131 occupies only a portion, namely a central portion, of the bottom and top side of the body 110, respectively. A secondary pattern 122, 132 is provided to the rest of the bottom and top side of the body 110, respectively. Naturally, further patterns are possible. The secondary pattern 122, 132 may be, as illustrated, an edge pattern knitted with a smooth and tight knit to provide for a relatively rigid yet comfortable edge. The tight knit in the edge pattern helps in maintaining the shape of the body 100. A border 123, 133 is shown in FIGS. 1A and 2A to designate the transition between the patterns 121, 122; 131, 132. In a physical piece the transition will be subtle. The border 123, 133 may take other shapes than the illustrated generally quadrilateral shape. The knit making up the secondary pattern 122, 132; 222, 232 may be varied in respect to the primary pattern 121, 131; 221, 231. The secondary pattern may include, for example, additional or substituting yarns for added elasticity. For example, the main yarn may be supplemented or replaced with a polyether-polyurea copolymer yarn. Additionally or alternatively, only the secondary pattern at the end 214 of the strap body may include a relatively softer knit.

Referring to FIG. 3A it may be noted that the knit on the edge 117, which defines the loop hole 116, may also feature a tighter knit than on the surrounding body 110. Additionally or alternatively the edge 117 may be knit with a different yarn than the surrounding body 110.

The same is applicable to the second strap part 200.

Referring to FIG. 1B and 2B it may be noted that the knit on the edge 235, which defines the eyelet 234, may also feature a tighter knit than on the surrounding body 210. Additionally or alternatively the edge 235 may be knit with a different yarn than the surrounding body 210, particularly more durable yarn than the main yarn or yarns making around the edge 235.

The strap body 110 is knitted from at least two yarns, namely from a first main yarn and from an interlooped reinforcing yarn. The first main yarn and the reinforcing yarn may be knitted into patterns as described above. The first main yarn may be a synthetic yarn, such as polyester, polyether-polyurea copolymer (sold under tradenames elastane, Lycra, or Spandex), polyurethane, or polyamide, or made from natural fibers, such as wool, or hemp, linen. The yarns can be spun, texturized, multifilament or monofilament.

The reinforcing yarn is interlocked with the first main yarn in the knit. The reinforcing yarn is a heat activated yarn that is fusible in lower temperatures than the first main yarn. According to one embodiment the reinforcing yarn has a melting point between 60 and 180° C. On the contrary, the melting point of the first main yarn, which may a different grade of polyester, may be about 260° C. An exemplary reinforcement yarn suitable for the present purpose is a fusible polyester yarn with a melting point of 60° C. Such yarns are commercially available under the trademark Grilon.

The reinforcing yarn content in the knit may be 50% or less, particularly 25 or less. According to a preferred embodiment the reinforcing yarn content in the knit is less than 25%.

After the strap body 110 is knit from the yarns, the reinforcing yarn may be activated by heating to or beyond the shrinking temperature of the reinforcing yarn so as to reduce the length of the reinforcing yarn. As the reinforcing yarn shrinks, it compresses the other yarns interlooped there with to create a reinforcement zone. The resulting knit is therefore irreversibly knitted. The heat activation may be performed with steam, hot air, infra-red rays, or a comparable heating method.

The strap body 110 may include one or more than one reinforcement zone for adjusting the rigidity properties of the strap body throughout or at selected sections of the strap body. In the illustrated example the reinforcement zone covers the entire body, i.e. reinforcement yarn is knitted into the entire body 110.

During said heat activating of the reinforcing yarn the strap body 110 may be heated to a temperature of the reinforcing yarn that is 30° C. or more above the melting point of the reinforcement yarn. The melting temperature of the reinforcing yarn is lower than that of the other yarn(s) making up the knit. The melting reinforcement yarn integrates the reinforcement yarn into the surrounding main yarn. The melting of the reinforcement yarn thus fuses the surrounding yarns together reinforcing the body 110.

Additionally or alternatively, the reinforcing yarn is simply hardened or cured by heat to a hardness that increases the inherent stiffness of the body 110.

During said heat activating of the reinforcing yarn the temperature is preferably kept below the melting point of the main yarn(s) to avoid distorting the shape and/or appearance of the knit.

According to a further embodiment, the knit making up the strap body 1a, 1b includes a second main yarn which is interlooped with the first main yarn and the reinforcing yarn. The second main yarn is may be made of a different material than the first main yarn and the reinforcing yarn. The second main yarn may, for example, be made of a coarser material than first main yarn. The cross-sectional shape of the second main yarn may be more angular, i.e. less round, than that of the first main yarn.

Further functional yarns in the knit are also possible. The knit may, for example, comprise silver yarns and/or reflective yarns. Silver yarns prevent the growth of bacteria, mold and/or fungi. Heat-activatable yarns may comprise heat-activated adhesive material having a melting point below that of the base yarn. Reflective yarns comprise reflective and/or retroreflective material.

The different patterns of the strap may be knitted with completely or predominantly different yarns. For example the inner layer 130 or only one pattern thereof may be knitted predominantly of the first main yarn, while the outer layer 120 or only one pattern thereof may be knitted predominantly of the second main yarn. According to a particular embodiment the façade pattern 121 may be knitted with polyamide and the skin contact pattern 131 may be knitted with polyester. Additionally or alternatively, the first and second main yarns may have different cross-sectional shapes with each other. While the first main yarn may have a relatively round or flat cross-section, the second main yarn may have a more angular cross-section.

According to an alternative embodiment, the main yarn material may be the same on both layers 120, 130 but the heat activation may be different. For example, only the reinforcing yarn embedded in the outer layer 120 may be heat activated.

The outer layer 120 may be harder than the inner layer 130, implying that Shore A hardness of the outer layer 120 is greater than that of the inner layer 130. Shore A hardness of the outer layer 120 may be at least 10%, preferably at least 15% greater than that of the inner layer 130. The outer layer 120 may be more durable/wear resistant than the inner layer 120. The difference may result from difference in main yarn material, knit pattern, heat treatment, or any combination of the above.

In addition or instead of the layers 120, 130, the strap body 110 may include zones, such as those featuring the different patterns 121, 122; 131, 121. The strap body 110 may include a central zone extending along the major dimension of the strap body and peripheral zones on either or both sides of the central zone. Similarly to the above described concept of different layers, the zones may have different properties, for example different material properties and/or physical properties, arising from different yarn materials used for the zones and/or different heat activation of the reinforcing yarn. The material property can be a mechanical property, for example elasticity, stiffness/flexibility, hardness/softness (Shore A hardness), durability or surface roughness, or optical property, e.g. reflectivity. The elasticity and/or softness of the edge zones may be greater than that of the central zone. The elastic modulus, particularly Young's modulus, of the central zone may be 10% or more, such as 15% or more, greater than that of the edge zones. The elastic modulus is the ratio of stress, below the proportional limit of the material, to the corresponding strain. The greater the elastic modulus, the stiffer the material, or the smaller the elastic strain that results from the application of a given stress. The elastic properties of the edge zones and the central zone can be adjusted by changing the density/number of yarns in the knit.

The width of the central zone may be 20 to 70%, such as 30 to 60% of the width of the strap body 110. The combined width the edge zones may be 30 to 80%, such as 40 to 70% of the width of the strap body 110. The edge zones may have equal width. The width of the strap 1 may be 22 mm.

In the manufacturing process the main yarn or yarns as well as the reinforcing yarn is fed from a reel of the 3D knitting machine. With the strap body 100 complete, accessories, such as buckles, spring bars, etc. are attached to the strap body 1a, 1b, where after the strap may be attached to a wearable device, such as a smart watch. Certain section or sections of the knit is heat activated by the heat activation processes mentioned above.

An additional layer of protective and/or decorative material may be laminated onto the strap body 110. Examples of such layers include a transfer print layer, fusible fabric layer, embroiled layer, a heat laminated layer, a silk screen printed layer, or another comparable additional textile membrane known in the field.

The bulk of the strap body 110 may vary between 700 and 1400 g/m², e.g. between 800 and 1100 g/m². The bulk of the strap body is preferably between 900 and 1000 g/m².

The proposed strap body 110 provides for a relatively stiff knitted structure which can preferably be handled with one hand, which is particularly useful in mating the watch parts in securing the device to the wrist, for example. The resulting stiffness about a bending axis, which is co-planar and transverse in respect to the major dimension of extension, is preferably such to withstand the weight of the strap body 110 about said bending axis without bending more than the width of the strap.

The two different zones and/or layers 120, 130 may have mutually different flexibility properties. The flexibility properties are observed in the major dimension in which the strap has the largest extension, i.e. the length of the strap. In particular, the second part 200 should have noticeable longitudinal flexibility. The flexibility should preferably be enough to stretch the strap by the distance of one or more eyelets 234.

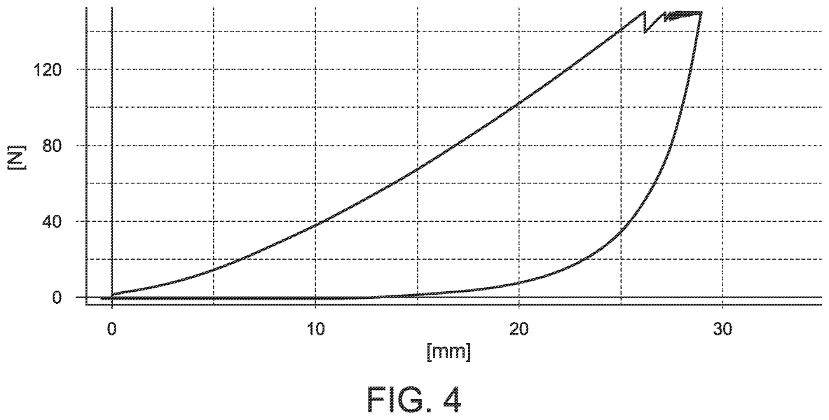

FIG. 4 shows a force vs displacement graph of an exemplary strap body 3D knitted from a polyester yarn reinforced with Grilon® reinforcement yarn to a width of 24 mm. As you can see, a force required to stretch the fabric of 5 mm is little over 10 N. It is advantageous to have the force required to impose an elastic displacement of 5 mm between 5 and 15 N for providing enough elasticity to align the buckle tang 152 of the first part 100 with the next available eyelet 234 of the second part 200 so as to keep the strap tightly against the user's body. Accordingly, the optical or other sensors housed in the wearable device may benefit from a reliable skin contact.

The herein described embodiments may be varied without departing from the general inventive concept.

For example, the buckle-eyelet construction could be replaced with a Velcro or other fastening construction.

Additionally or alternatively, the strap may comprise only a single strap part, in a heart rate sensor strap application, for example.

Additionally or alternatively, the strap body may comprise at least one pocket or void for receiving a measuring sensor, e.g. a heart rate sensor.

Additionally or alternatively, the thickness of strap body 110 may be varied across different dimensions. For example, more material and/or volume may be knitted into the ends 113, 114 of the body 110. The additional material and/or volume will promote the stiffness of the body 110 in withstanding the weight of the terminal device. 3D knitting allows for building volume to the strap ends 113 and 213 at the watch case attachment points. This makes it possible to make fluent shapes where strap attaches to casing. There would be no sharp edges or crevices that can hook to bushes, for example, when outdoor sporting.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### LIST OF REFERENCE NUMERALS

| No. | Feature |
|---|---|
| 100 | first part |
| 110 | strap body |
| 111 | first side |
| 112 | second side |
| 113 | first end |
| 114 | second end |
| 115 | release hole |
| 116 | loop hole |
| 117 | loop edge |
| 120 | outer layer |
| 121 | façade pattern |
| 122 | secondary pattern |
| 123 | border |
| 124 | pore |
| 130 | inner layer |
| 131 | skin contact pattern |
| 132 | second pattern |
| 133 | border |
| 140 | spring bar |
| 150 | buckle |
| 151 | frame |
| 152 | tang |
| 153 | pin |
| 160 | loop |
| 200 | second part |
| 210 | strap body |
| 211 | first side |
| 212 | second side |
| 213 | first end |
| 214 | second end |
| 215 | release hole |
| 216 | loop hole |
| 217 | loop edge |
| 220 | outer layer |
| 221 | façade pattern |
| 222 | secondary pattern |
| 223 | border |
| 230 | inner layer |
| 231 | skin contact pattern |
| 232 | second pattern |
| 233 | border |
| 234 | eyelet |
| 235 | edge |
| 240 | spring bar |

1. A wearable device comprising:
an optical sensor housed in the wearable device, and
a strap configured to be attached to the wearable device so as to be worn on a user, wherein the strap comprises a strap body knitted from at least a first main yarn and a heat activated reinforcing yarn, wherein the strap body comprises at least one reinforcement zone, which covers only a portion of the strap body and in which reinforcement zone the properties of the reinforcement yarn have been altered through application of heat for varied flexibility properties across different sections of the strap.

2. The wearable device according to claim 1, wherein the strap body is three-dimensionally machine-knitted.

3. The wearable device according to claim 1, wherein the reinforcing yarn is a fusible yarn.

4. The wearable device according to claim 1, wherein the reinforcing yarn has a melting point below that of the other yarns making up the strap body.

5. The wearable device according to claim 1, wherein the reinforcing yarn has a melting temperature between 60 and 180° C.

6. The wearable device according to claim 1, wherein the reinforcing yarn content in the knit is 50% or less, particularly 25% or less, preferably less than 25%.

7. The wearable device according to claim 1, wherein the at least one reinforcement zone extends throughout the strap body.

8. The wearable device according to claim 1, wherein the reinforcement zone comprises compression of the reinforcing yarn, through heat activated reduction in length, imposed on the main yarn interlooped with the reinforcing yarn.

9. The wearable device according to claim 1, wherein the reinforcement zone comprises fusion of the reinforcing yarn into the first main yarn through heat activated melting of the reinforcing yarn.

10. The wearable device according to claim 1, wherein the strap comprises a major dimension, wherein the strap body has a stiffness about a bending axis, which is co-planar and transverse in respect to the major dimension of extension, to withstand the weight of the strap body about said bending axis without bending.

11. The wearable device according to claim 1, wherein:
the strap (1) comprises:
a major dimension,
a minor dimension, which is transverse to the major dimension, and
a thickness, which is transverse to both the major dimension and the minor dimension, and wherein
the strap has a flexibility along the major dimension that is greater flexibility of the strap along the minor dimension.

12. The wearable device according to claim 11, wherein:
the strap body comprises a plurality of eyelets provided along the strap body, and wherein
the flexibility of the strap along the major dimension is such to allow for elastic deformation of the strap body by at least the distance between successive eyelets.

13. The wearable device according to claim 1, wherein the bulk of the strap body is between 700 and 1400 g/m².

14. The wearable device according to claim 1, wherein the knit forming the strap body of the strap comprises a second main yarn interlooped with the first main yarn and the reinforcing yarn.

15. The wearable device according to claim 14, wherein:
the second main yarn is made of a different material than the first main yarn and the reinforcing yarn,
the first main yarn has a different, e.g. rounder, cross-sectional shape than the second main yarn,
the second main yarn has a greater durometer value than the first main yarn, or
any combination of the above.

16. The wearable device according to claim 1, wherein the strap body comprises:
an inner layer for placement against user's skin during use, which inner layer:
is knitted predominantly of the first main yarn,
is knitted with a skin contact pattern, or
is knitted predominantly of the first main yarn into a skin contact pattern and
an outer layer for placement away from the user's skin during use, which outer layer:
is knitted predominantly of a/the second main yarn,
is knitted with a façade pattern or
is knitted predominantly of a/the second main yarn into façade pattern.

17. The wearable device according to claim 16, wherein:
the façade pattern is more porous than the skin contact pattern,
the façade pattern comprises visible pores,
the skin contact pattern is smoother than the façade pattern, or
any combination of the above.

18. The wearable device according to claim 1, wherein the strap comprises two edge zones at the edges of the strap extending in an longitudinal direction of the strap, and a central zone located between the edge zones, and that the elastic modulus of the central zone is greater than that of the edge zones.

19. The wearable device according to claim 1, wherein:
the strap body comprises a transverse loop hole at a first end of the strap body,
a transverse buckle hole at a second end of the strap body,
the material or pattern of the yarn defining the transverse hole is stiffer than the material of the yarn forming other parts of the strap body, or
any combination of the above.

20. The wearable device according to claim 19, wherein the strap comprises:
a first part comprising:
a spring bar provided to the transverse loop hole, and
a buckle attached to the transverse buckle hole, and
a second part comprises:
a spring bar provided to the transverse loop hole, and
a plurality of eyelets provided along the second part, whereby the second part is configured to cooperate with the buckle.

21. A method of producing the strap of the wearable device according to claim 1, the method comprising:
providing a first main yarn;
providing a heat activated reinforcing yarn;
three-dimensionally knitting the first main yarn and reinforcing yarn into a one piece strap body in a three-dimensional knitting machine, and by
increasing the rigidity of the strap body at the reinforcement zone by heating the reinforcing yarn to or beyond the point of heat activation for varied flexibility properties across different sections of the strap, which reinforcement zone covers only a portion of the strap body.

22. The method according to claim 21, wherein the step of increasing the rigidity of the strap body comprises irreversible changing of the physical properties of the reinforcement yarn.

23. The method according to claim 21, wherein the step of increasing the rigidity of the strap body comprises fusing the reinforcing yarn into the interlooped main yarn.

24. The method according to claim 21, wherein the method further comprises providing a second main yarn and knitting the second main yarn into the knit forming the strap body.

25. The method according to claim 21, wherein the knitting step comprises knitting two or layers having different patterns or comprising predominantly different main yarns or both having different patterns and comprising predominantly different main yarns.

26. The method according to claim 21, wherein the method further comprises installing hardware to the strap body prior to the heat activation step.
